(19) United States
(12) Patent Application Publication
(10) Pub. No.: US 2022/0299461 A1
(43) Pub. Date: Sep. 22, 2022

(54) Heater Meander

(71) Applicant: TDK Corporation, Tokyo (JP)

(72) Inventors: Andreas Barbul, München (DE); Matthias König, München (DE)

(21) Appl. No.: 17/692,990

(22) Filed: Mar. 11, 2022

(30) Foreign Application Priority Data
Mar. 16, 2021 (DE) 102021106388.4

(51) Int. Cl. G01N 27/16; G01N 27/18

(52) U.S. Cl. CPC G01N 27/16; G01N 27/18; H05B 2203/037; H05B 2203/003

(57) ABSTRACT

A heat meander is disclosed. In an embodiment a heater meander includes a meander structure, wherein central meander lines of the meander structure are thicker than meander lines in an outer area of the meander structure.

### CROSS-REFERENCE TO RELATED APPLICATIONS

This application claims priority to German Patent Application No. 102021106388.4, filed on Mar. 16, 2021, which application is hereby incorporated herein by reference.

### TECHNICAL FIELD

In state of art gas concentration sensors (sensors for measuring a gas concentration) such as contact combustion type sensors or heat conduction type sensors the gas mixture to be analyzed is heated to a predetermined temperature by a resistor heater.

### BACKGROUND

In order to allow a good heat transition between the resistor heater and the gas, the resistor heater can be configured as a heater meander with a meandering shape.

The US patent application US 2019/353607 A1 discloses an example of a gas concentration sensor comprising both a contact combustion type sensors and a heat conduction type sensor each comprising a meandering resistor heater.

The heat dissipation of a meander structure has usually a negative gradient from a central area to an outer area of the meander structure. Especially for gas concentration sensors it is crucial to significantly decrease the gradient to be able to improve the sensor's sensitivity.

### SUMMARY

Embodiments provide a heater meander comprising a meander structure, wherein the thickness of central meanders of the meander structure is bigger than the thickness of meanders at the terminations of the meander structure.

The heater meander may be configured as a resistor heater.

In this case, the meander structure serves as an electrical conductor. The heater meander heats up due to its electrical resistance. The meander structure increases heat transfer from the heat meander to its environment, in particular to a gas.

A meander structure with an increased thickness of the central meanders allows homogeneous heating in its central area. This pattern of meanders is designated as thick meander pattern. Adjacent to the central area, where the meanders are thicker than in the outer area, a thermistor for measuring the temperature of a gas can be arranged. The area in which the thermistor is arranged is designated as a sensing area.

In this case, the gas in the whole sensing area is heated homogeneously since heat is homogeneously transferred from the heater meander to the gas. Thus, the measurements of gas temperature and concentration are more accurate, wherein the gas concentration is calculated from the measured temperature.

The thermistor may either be an NTC or a PTC thermistor comprising e.g. one or more materials selected from a composite metal oxide, amorphous silicon, polysilicon, or germanium.

Embodiments further provide a heater meander comprising a meander structure, wherein the meanders form an alternating pattern of alternating thin and thick meander lines.

A meander structure with the alternating pattern allows also homogeneous heating. When a thermistor is arranged adjacent to the meander structure, the gas in the whole sensing area is again heated homogeneously since heat is homogeneously transferred from the heater meander to the gas. Thus, the measurements of gas temperature and concentration are more accurate.

Additionally, an alternating pattern of meanders results in lower heat dissipation of the heater meander compared to a pattern in which the meanders are shaped homogeneously.

Therefore, the alternating pattern may be preferably used when heating to smaller temperatures is required.

In a preferable embodiment, both meander structures, which are described before, are combined.

The meander structure may be configured with an alternating pattern as described before. Additionally, the meanders in the central area of the meander structure may be thicker than in the outer area.

In general, if the thickness of a meander line is large, the electrical resistance is comparable small. As a result less electrical power is consumed and the electrical voltage drops less at these points.

The heater meander according to one of the before mentioned aspects comprises a meander structure which is configured to obtain a homogeneous temperature field in a sensing area.

Further embodiments provide a gas concentration sensor comprising a heater meander according to one of the before mentioned aspects.

In embodiments, the gas concentration sensor is a contact combustion type sensor or a heat conduction type sensor.

By applying the described geometric shapes to the heater meander the sensing efficiency of a gas concentration sensor can be increased as described above.

In particular, in an embodiment the gas concentration sensor comprises at least to sensor units.

In particular, the first unit may comprise a first thermistor and a first heating resistor comprising a first heater meander, and the second unit may comprise a second thermistor and a second heating resistor comprising a second heater meander. The first thermistor is covered with a catalyst and the second thermistor is covered with a dummy catalyst.

The dummy catalyst does not contain a catalytic metal but is provided for matching the heat capacity of the first thermistor and the second thermistor.

When being heated to a predetermined temperature by the first heater resistor, the catalyst accelerates a reaction, e.g. a combustion, between a gas to be detected and a reaction gas in the atmosphere. Reaction heat generated at this time is conducted to the first thermistor to change the resistance value thereof. On the other hand, the dummy catalyst does not accelerate a reaction of the gas even when being heated to a predetermined temperature by the second heater resistor, so that the resistance value of the second thermistor reflects only heating by the second heater resistor.

The first unit is designated as measuring sensor unit and the second unit is designated as reference sensor unit.

A detection signal output from a connection point between the first and second thermistors has a level corresponding to the concentration of the gas to be measured in a measured environmental atmosphere.

Depending on a configuration of the gas concentration sensor, the different shaped heater meanders, as described before, may be used in different sensor units. For example, the alternating pattern may be applied in a reference sensor unit and the thick meander pattern may be used in a measuring sensor unit.

### BRIEF DESCRIPTION OF THE DRAWINGS

In the following the invention is exemplarily described by using figures. The invention is not limited to the described examples.

FIG. 1 shows a simulation of the temperature field of a first central meander area;

FIG. 2 shows a simulation of the temperature field of a second central meander area;

FIG. 3 shows a simulation of the temperature field of a third central meander area;

FIG. 4 shows a simulation of the temperature field of a fourth central meander area;

FIG. 5 shows a simulation of the temperature field of a fifth central meander area;

FIG. 6 shows a first embodiment of an improved meander structure;

FIG. 7 shows a second embodiment of an improved meander structure; and

FIG. 8 shows a schematic diagram of an embodiment of a gas concentration sensor.

### DETAILED DESCRIPTION OF ILLUSTRATIVE EMBODIMENTS

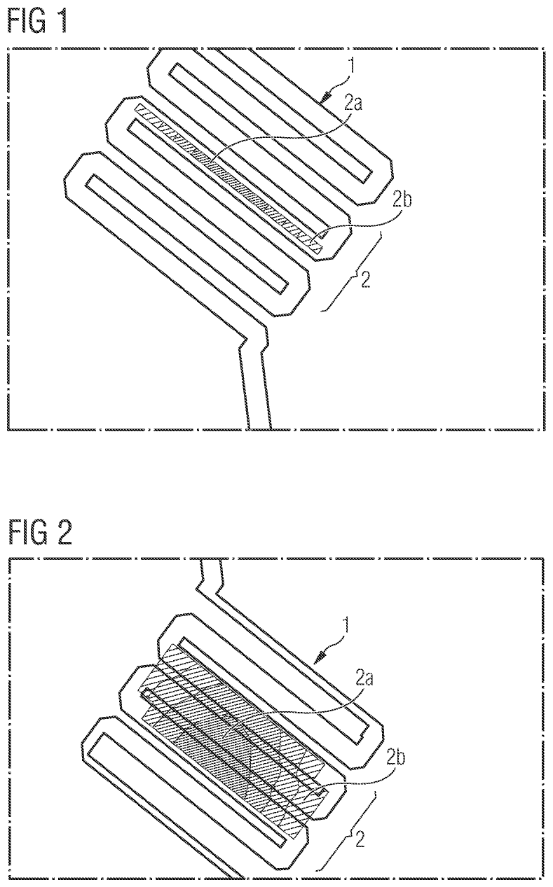

In the FIGS. 1 to 7 different patterns of meander structures and different thicknesses of meander lines of a resistor heater meander 1 are shown. The resulting temperature fields of adjacent sensing areas are simulated and are shown as shaded areas. A suiting meander geometry provides a more homogeneous temperature field in the sensing area of a gas concentration sensor.

The sensing area is the area at a surface of the meander structure next to which a thermistor for gas temperature measurement is arranged.

The thermistor may be the thermistor of a measuring sensor unit or a reference sensor unit. The functionality of the two units are described later. In general, thermistors in reference sensor units are smaller to obtain a temperature that is as uniform as possible. Thermistors in measuring sensor units are larger to improve their detection properties.

All temperatures in the figures are given in Kelvin (K). The maximum and minimum temperatures are shown. Different types of shading 2a and 2b symbolise different temperatures. A finer shaded area 2a symbolises an area of higher temperature and the area 2b is an area of lower temperature.

In a meander structure shown in FIG. 1, which is not an embodiment of the invention, the meander lines have a uniform small thickness.

Thus, the gradient of a temperature of a gas in the sensing area is large. In the simulated example of a temperature field of the sensing area shown in FIG. 1, a maximum temperature in the center of the meander amounts 648 K and a minimum temperature in an outer area of the meander amounts 610 K.

In the embodiment shown in FIG. 2, the thickness of the meanders in a central area 2 of the meander structure is slightly increased. As a result, the temperature gradient decreases.

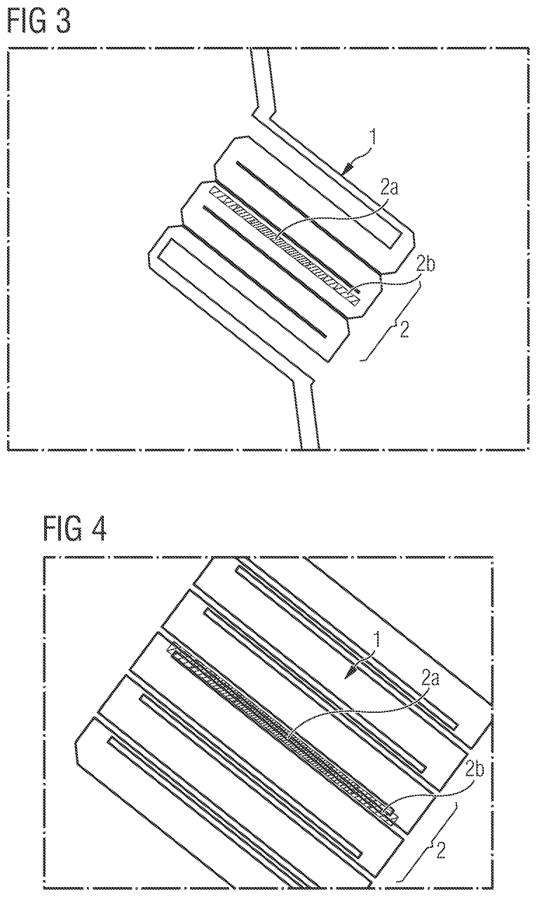

In the embodiment shown in FIG. 3, the thickness of the meanders in a central area 2 is further increased. As a result, the temperature gradient decreases significantly.

In FIGS. 1 and 3 sensing fields of small thermistors as used in reference sensor units are shown, whereas in FIG. 2 a large thermistor as used in measuring sensor units is shown. In further examples, the embodiment of a meander structure shown in FIG. 2 may also be used with larger thermistors as used in measuring sensor units and the embodiment of a meander structure shown in FIG. 3 may also be used with smaller thermistors as used in reference sensor units.

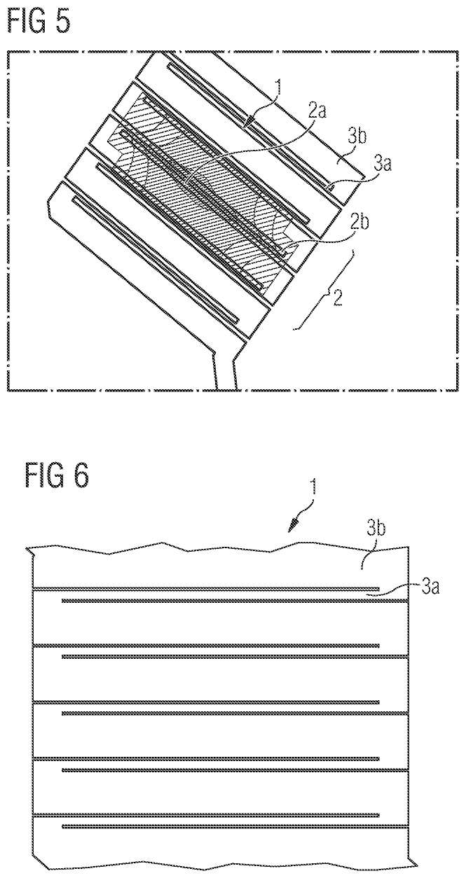

In the embodiments shown in FIGS. 4 and 5 a structure with a pattern of alternating thin and thick meander lines 3a and 3b is shown. In this case, temperature gradient in a sensing area decreases significantly.

In FIG. 4 a simulation of the temperature field in a small sensing area of a reference sensor unit is shown and in FIG. 5a large sensing area of a measuring sensor unit is shown.

Furthermore, a combination of both described effects, thicker meander lines in a central area 2 and alternating pattern of meander lines 3a and 3b, is possible to further decrease the temperature gradient in the sensing area.

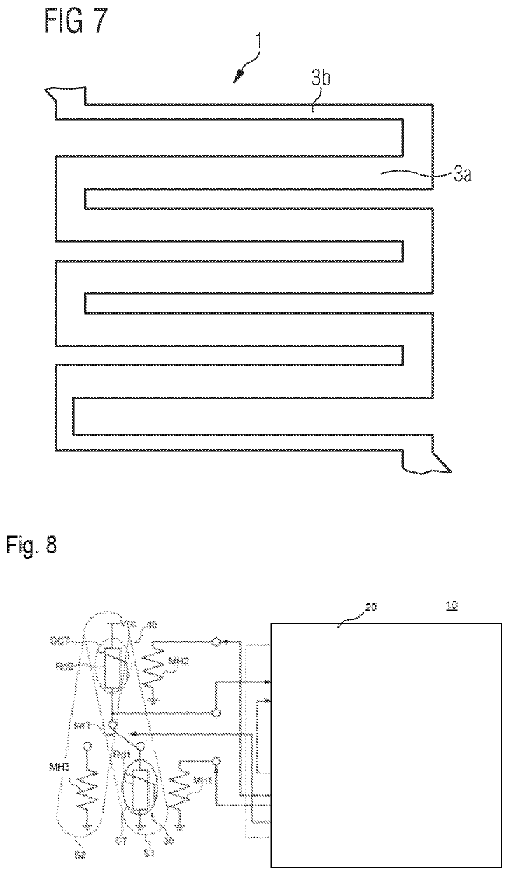

In particular, embodiments of meander structures with the meander patters shown in the FIGS. 6 and 7 are preferable.

FIG. 6 shows an example of a meander structure with a pattern of alternating thick and thin meander lines 3a and 3b.

FIG. 7 shows an example of a meander structure with thick meander lines 3b in a central area 2 and thin meander lines 3a in an outer area.

In both embodiments which are shown in FIGS. 6 and 7, each corner or edge of a meander line may have additional chamfers, which are not shown in the figures. Further, each meander line may have additional narrowings to optimize the shape and to achieve optimized heat conduction properties of the meander.

In an embodiment, a gas concentration sensor comprises a measuring sensor unit with a first resistor heater meander. A catalyst in the measuring sensor unit accelerates a reaction, e.g. combustion, between e.g. CO gas to be detected and e.g. 02 gas as a reaction gas in the atmosphere to generate CO₂ gas during operation of the sensor. Reaction heat generated at this time is conducted to a first thermistor adjacent to a sensing area of the first resistor heater meander in the measuring sensor unit to change the resistance value of the first thermistor.

In the same embodiment, the gas concentration sensor comprises a reference sensor unit with a second resistor heater meander. A dummy catalyst in the reference sensor unit does not accelerate the combustion of the gas to be detected even when being heated to a predetermined temperature by the second resistor heater. Thus, the resistance value of the second thermistor reflects only heating by the second resistor heater meander. The concentration of the gas to be detected can be calculated from the different signals of the measuring sensor unit and the reference sensor unit.

In a further embodiment, a gas concentration sensor comprises a first sensor part that is configured to detect a concentration of a mixture of a first gas and a second gas; and the gas concentration sensor comprises a second sensor part having a higher detection sensitivity with respect to the second gas than with respect to the first gas, and a signal processing circuit that subtracts a concentration of the second gas detected by the second sensor part from a mixture concentration detected by the first sensor part to derive a concentration of the first gas.

In an embodiment, the first and second gases are combustible gases, and the first sensor part is a contact combustion type sensor.

In an embodiment, the second sensor part is a heat conduction type sensor. This configuration is advantageous, if the heat conductivity of the first gas is e.g. closer to the heat conductivity of a measuring atmosphere than the heat conductivity of the second gas.

In the following an embodiment of a gas concentration sensor comprising a resistor heater meander is exemplarily described. The invention is not limited to the described example.

The exemplary gas concentration sensor 10 shown in FIG. 8 includes a first sensor part S1 that is configured to detect the concentration of the mixture of first gas and second gas, a second sensor part S2 having higher detection sensitivity with respect to the second gas than with respect to the first gas, and a signal processing circuit 20 that is configured to subtract the concentration of the second gas detected by the second sensor part from the mixture concentration detected by the first sensor part to derive the concentration of the first gas.

The concentration of the second gas detected by the second sensor part S2 can be subtracted from the concentration of the mixture concentration detected by the first sensor part S1 by the signal processing circuit 10, so that it is possible to cancel the influence of the second gas which may be a miscellaneous gas mixture to thereby work out a correct value of concentration of the first gas to be detected.

In an example the first and second gases may be combustible gases and the first sensor part S1 is a contact combustion type sensor, and the first gas is closer in heat conductivity to the measuring environmental atmosphere than the second gas and the second sensor part S2 is a heat conduction type sensor. With this configuration, it is possible to reduce measurement errors caused due to the presence of a second gas which may be a mixture of miscellaneous combustible gases. The first gas is, e.g., CO gas, and the second gas is, e.g., ethanol, acetic acid, or an organic deodorant.

It is preferable in this embodiment that the first sensor part S1 includes a measuring sensor unit 30 comprising a first resistor heater meander MH1, a first thermistor Rd1 and a catalyst CT disposed near the first thermistor Rd1 and further includes aa reference sensor unit 40 comprising a second resistor heater meander MH2, a second thermistor Rd2, which does not comprise a catalyst or which comprises only a dummy catalyst DCT near the second thermistor Rd2. With this configuration, it is possible to accelerate combustion of the gas to be detected in the measuring sensor unit 30 by the catalyst CT.

In this embodiment, the second sensor part S2 may include a further measuring sensor unit comprising a third resistor heater meander MH3. The reference sensor unit 40 may be shared between the first and the second sensor parts S1 and S2.

The first sensor part S1 may output a first detection signal and the second sensor part S2 may output a second detection signal. With this configuration, the number of required elements can be reduced. The second sensor part 40 may be either connected to the first sensor part S1 or the second sensor part S2 by a switch sw1.

With this configuration, it is possible to reduce measurement errors due to aging of the thermistor, a change in environmental temperature, or the presence of non-combustible second gas.

Alternatively, the gas concentration sensor may have a configuration in which the first sensor part S1 includes two sensor units and the second sensor part S2 includes two sensor units, a reference sensor unit and a measuring sensor unit each.

With this configuration, measurement by the first sensor part S1 and measurement by the second sensor part S2 may be executed synchronously (at the same time) or asynchronously (at different times).

With this configuration, it is also possible to reduce measurement error due to aging of the thermistor, a change in environmental temperature, or presence of non-combustible miscellaneous gases.

The signal processing circuit 20 preferably determines the type of the second gas based on the detection signal of the first sensor part S1 during operation. In this case, the signal processing circuit 20 can determine the type of the second gas from the rising waveform of the first detection signal. Further, the signal processing circuit 20 may determine the presence/absence of the first gas according to the inclination of the first detection signal.

The signal processing circuit 20 may calculate the concentration of the first gas in a predetermined detection cycle, and the detection cycle may be reduced when the concentration of the second gas detected by the second sensor part S2 exceeds a predetermined value.

The signal processing circuit preferably corrects the difference between detection sensitivity with respect to the second gas by the first sensor part S1 and detection sensitivity with respect to the second gas by the second sensor part S2. Thus, it is possible to accurately cancel the influence of the second gas.

While this invention has been described with reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A heater meander comprising:
a meander structure,
wherein central meander lines of the meander structure are thicker than meander lines in an outer area of the meander structure.

2. The heater meander according to claim 1, wherein the meander structure is configured to obtain a homogeneous temperature field in a sensing area.

3. The heater meander according to claim 1, wherein the heater meander is a resistor heater

4. A heater meander comprising:
a meander structure, wherein meander lines form a pattern of alternating thin and thick meander lines.

5. The heater meander according to claim 4, wherein the meander structure is configured to obtain a homogeneous temperature field in a sensing area.

6. The heater meander according to claim 4, wherein the heater meander is a resistor heater.

7. A gas concentration sensor comprising:
a heater meander comprising a meander structure,
wherein central meander lines of the meander structure are thicker than meander lines in an outer area of a meander structure, or
wherein the meander lines form a pattern of alternating thin and thick meander lines.

8. The gas concentration sensor according to claim 7, further comprising a thermistor arranged adjacent to a sensing area of the heater meander.

9. The gas concentration sensor according to claim 8, wherein the gas concentration sensor comprises two sensor units, wherein a first sensor unit comprises a first thermistor and a first heater meander and a second sensor unit comprises a second thermistor and a second heater meander.

10. The gas concentration sensor according to claim 9, wherein the first thermistor is covered with a catalyst and the second thermistor is covered with a dummy catalyst.

11. The gas concentration sensor according to claim 10,
wherein one of the two sensor units comprises the heater meander,
wherein the central meander lines of the meander structure are thicker than the meander lines in the outer area of the meander structure,
wherein the other one of the two sensor units comprises the heater meander, and
wherein the meander lines form the pattern of alternating thin and thick meander lines.
